(19) United States
(12) Patent Application Publication
(10) Pub. No.: US 2022/0256845 A1
(43) Pub. Date: Aug. 18, 2022

(54) ANTIBACTERIAL OR ANTIFUNGAL COMPOSITION

(71) Applicants: INDUSTRY-ACADEMIC COOPERATION FOUND'N, YONSEI UNIV, Seoul (KR); THE REGENTS OF THE UNIVERSITY OF MICHIGAN, Seoul (KR)

(72) Inventors: Sung-Hwan Choi, Seoul (KR); Jae Sung Kwon, Seoul (KR); Jie Jin, Seoul (KR); Kenichi Kuroda, Saline, MI (US)

(21) Appl. No.: 17/672,651

(22) Filed: Feb. 15, 2022

(30) Foreign Application Priority Data
Feb. 16, 2021 (KR) 10-2021-0020380

(51) Int. Cl. A01N 37/44; A61L 27/34; A61L 31/10; A61L 31/16; A61L 27/54

(52) U.S. Cl. CPC A01N 37/44; A61L 27/34; A61L 31/10; A61L 2300/21; A61L 27/54; A61L 2300/404; A61L 31/16

(57) ABSTRACT

The present invention relates to an antibacterial or antifungal composition containing methoxyethyl acrylate.

### CROSS-REFERENCE TO RELATED APPLICATIONS

This application claims the benefit of Korean Patent Application no. 10-2021-0020380, filed Feb. 16, 2021, which is hereby incorporated herein by reference in its entirety.

### BACKGROUND

### 1. Technical Field

The present invention relates to a composition having improved antibacterial or antifungal activity.

### 2. Related Art

Various polymer resins have been used as denture base acrylic resins in dentistry. Due to their properties, various polymer resins have been used in the biomedical field as bone cements in implant surgery. For biomedical applications, the evaluation of biological properties such as cytotoxicity, in vitro and in vivo biocompatibility, and antimicrobial effects provide essential information about the interactions between materials and biological systems, which can be used for the development of new materials or new application programs. In addition, the use of additives to improve properties requires the modification of synthesis methods and the evaluation of new properties relating to the resulting material.

Meanwhile, silver nanoparticles (AgNPs) have been used in the biomedical field as an antimicrobial agent to prevent infections or colonization of biomedical devices by pathogenic microorganisms. In dentistry, AgNPs have been used to improve the mechanical properties of restorative materials and to promote colonization of dental prostheses' surfaces. However, AgNPs are problematic in terms of biocompatibility, such as causing severe cell transformation.

Accordingly, there is a demand for highly stable materials which have adequate hardness to withstand changes in temperature, acidity, pressure and humidity in the human body, are difficult for bacteria and fungi to grow, and do not elute substances having adverse effects on the human body, such as allergy and endocrine disturbance.

The “Related Art” section has been written to facilitate understanding of the present invention. It should not be understood as an admission that the matters described in the “Related Art” section exist as prior art.

### SUMMARY

An object of the present invention is to provide a composition containing methoxyethyl acrylate having improved antibacterial or antifungal activity and improved mechanical properties. Another object of the present invention is to provide a composition that may be applied to a medical device having antibacterial or antifungal activity together with antifouling activity.

However, objects of the present invention are not limited to the objects mentioned above, and other objects not mentioned herein may be clearly understood by those of ordinary skill in the art from the following description.

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

In the present invention, MEA is an abbreviation for methoxyethyl acrylate. Preferably, in the present invention, MEA is an abbreviation for 2-methoxyethyl acrylate. MEA used in an example of the present invention is one commercially available from Sigma (molecular weight=130.14). In the present invention, PMEA is an abbreviation for poly(2-methoxyethyl acrylate). In the present invention, PMMA is an abbreviation for poly(methyl methacrylate).

As used herein, the term “elastic modulus” refers to the ratio of stress to strain in the elastic range of any material. The elastic modulus indicates the slope of the straight section in a stress-strain curve representing the relationship between stress and strain. A material with a high elastic modulus is a stiff material, and a material with a low elastic modulus is a flexible material.

As used herein, the term “hardness” refers to the degree of hardness of the material surface. Methods for measuring hardness by resistance to indentation include Brinell, Rockwell, Vickers, and Knoop hardness measurement methods. Methods for measuring hardness by rebound include Shore hardness measurement, and methods for measuring hardness by resistance to scratching include Mohs hardness measurement.

As used herein, the term “wettability” refers to the degree to which a solid material is wetted by a liquid material.

As used herein, the term “contact angle” is a measure of the wettability of a solid by a liquid and refers to an angle formed between a liquid droplet placed on the solid surface and the solid surface. In general, the contact angle decreases as the surface energy of the solid increases and as the surface tension of the liquid decreases. A material with a large contact angle with water is considered hydrophobic, and a material with a small contact angle with water is considered hydrophilic.

As used herein, the term “polydispersity index (PDI)” refers to an index indicating a measure of non-uniformity in molecular characteristics, such as the mass, size, shape, and stereoregularity of molecules or particles, in a solution. In the present invention, the polydispersity index of the polymer is expressed as Dm. The polydispersity index of the polymer is defined as the ratio of the weight-average molar mass to the number-average molar mass.

In the present invention, gel permeation chromatography (GPC) is a method of separating materials by molecular weight difference using a column packed with porous gel. In an example of the present invention, using the GPC method, the number-average molecular weight (Mn), weight-average molecular weight (Mw) and polydispersity index (Dm) were measured based on poly(methyl methacrylate).

To achieve the above objects, the present invention provides an antibacterial and antifungal composition containing methoxyethyl acrylate.

In one embodiment of the present invention, the composition further contains poly(methyl methacrylate). In another embodiment of the present invention, the methoxyethyl acrylate may have a number-average molecular weight (Mn) of 1,000 to 100,000. In another embodiment of the present invention, the methoxyethyl acrylate may have a number-average molecular weight (Mn) of 1,050 to 90,000. In another embodiment of the present invention, the number-average molecular weight (Mn) of the methoxyethyl acrylate may be greater than the number-average molecular weight (Mn) of MEA and may be 100,000 or less. In another embodiment of the present invention, the methoxyethyl acrylate may have a weight-average molecular weight (Mw) of 1,500 to 400,000. In another embodiment of the present invention, the methoxyethyl acrylate may have a weight-average molecular weight (Mw) of 1,500 to 350,000. In another embodiment of the present invention, the weight-average molecular weight (Mw) of the methoxyethyl acrylate may be greater than the weight average molecular weight (Mw) of MEA, and may be 400,000 or less. In another embodiment of the present invention, the methoxyethyl acrylate may have a polydispersity index of 1.1 to 3.0. In another embodiment of the present invention, the methoxyethyl acrylate may have a polydispersity index of 1.2 to 2.5. In another embodiment of the present invention, the methoxyethyl acrylate may have a polydispersity index of 1.3 to 2.0. In another embodiment of the present invention, the polydispersity index of the methoxyethyl acrylate may be greater than the polydispersity index of MEA, and may be 3.0 or less. In another embodiment of the present invention, the poly(methyl methacrylate) is a mixture of methyl methacrylate powder and methyl methacrylate liquid. In another embodiment of the present invention, the composition contains, based on the total weight of the composition, 42 to 59.8 wt % of methyl methacrylate powder, 30 to 39.8 wt % of methyl methacrylate liquid, and 1 to 20 wt % of methoxyethyl acrylate. In another embodiment of the present invention, the composition contains, based on the total weight of the composition, 45 to 59.2 wt % of methyl methacrylate powder, 33 to 39.5 wt % of methyl methacrylate liquid, and 1.5 to 18 wt % of methoxyethyl acrylate. In another embodiment of the present invention, the composition has antibacterial activity against at least one selected from the group consisting of Streptococcus mutans, Streptococcus sobrinus, Streptococcus sanguis, Streptococcus minor, Lactbacillus casei, Lactbacillus acidophilus, Porphyromonas gingivalis, Treponema denticola, Actinomyces naeslundii, Veillonella parvula, Actinomyces viscosus, and Actinomyces naeslundii. In another embodiment of the present invention, the composition has antifungal activity against at least one selected from the group consisting of Candida albicans, Escherichia coli, Staphyloccoccus aureus, Pseudomonas aeruginosa, and Aspergillus niger. In another embodiment of the present invention, the composition may be used for an internal restorative material, a temporary dental restorative material, a permanent dental restorative material, a pediatric dental restorative material, dentures, a dental implant, a mouthpiece, an occlusal stabilization splint, a night guard, an intraoral appliance, an activator, a snoring device, or an in vitro appliance. In another embodiment of the present invention, the composition has an effect of reducing the thickness of biofilm thereon by 60% to 70% compared to a control. In another embodiment of the present invention, the composition has an effect of reducing the biomass density of biofilm thereon by 70% to 80% compared to a control. In another embodiment of the present invention, the composition further contains at least one selected from the group consisting of a stabilizer, a flame retardant, an antistatic agent, a softener, a reinforcing material, a filler, a fluorescent whitening agent, a lubricant, an inclusion reducer, a polycondensation catalyst, an antifoaming agent, an emulsifier, a thickener, and fragrances. In another embodiment of the present invention, the composition further contains an adhesive material. In another embodiment of the present invention, the adhesive material is at least one selected from the group consisting of hydroxypropyl methylcellulose, hydroxyethyl cellulose, hydroxypropyl cellulose, polyvinyl alcohol, polyvinyl pyrrolidone, carbomer, and polyvinyl acetate resins.

In another embodiment of the present invention, when PMEA in the composition according to the present invention has a number-average molecular weight (Mn) of 1,000 to 100,000 as measured by gel permeation chromatography, it is referred to as low-molecular-weight PMEA. In another embodiment of the present invention, the low-molecular-weight PMEA has a number-average molecular weight (Mn) of 1,050 to 90,000 as measured by gel permeation chromatography. In another embodiment of the present invention, the low-molecular-weight PMEA has a number-average molecular weight (Mn) of 1,050 to 8,000 as measured by gel permeation chromatography for the composition according to the present invention. In another embodiment of the present invention, the low-molecular-weight PMEA has a number-average molecular weight (Mn) of 1,100 to 80,000 as measured by gel permeation chromatography for the composition according to the present invention. In another embodiment of the present invention, the low-molecular-weight PMEA has a number-average molecular weight (Mn) of 1,100 to 20,000 as measured by gel permeation chromatography for the composition according to the present invention. In another embodiment of the present invention, when the number-average molecular weight (Mn) of PMEA in the composition according to the present invention, measured by gel permeation chromatography (GPC), is greater than the number-average molecular weight (Mn) of MEA, measured by GPC according to the present invention, but is 100,000 or less, the PMEA is referred to as low-molecular-weight PMEA. In another embodiment of the present invention, when the number-average molecular weight (Mn) of PMEA in the composition according to the present invention, measured by gel permeation chromatography (GPC), is greater than the weight-average molecular weight (Mw) of MEA, measured by GPC according to the present invention, but is 400,000 or less, the PMEA is referred to as low-molecular-weight PMEA. In another embodiment of the present invention, when the weight-average molecular weight (Mw) of PMEA in the composition according to the present invention, measured by gel permeation chromatography (GPC), is 1,500 to 400,000, the PMEA is referred to as low-molecular-weight PMEA. In another embodiment of the present invention, the low-molecular-weight PMEA has a weight-average molecular weight (Mw) of 1,500 to 350,000 as measured by gel permeation chromatography. In another embodiment of the present invention, the low-molecular-weight PMEA has a weight-average molecular weight (Mw) of 1,700 to 300,000 as measured by gel permeation chromatography. In another embodiment of the present invention, the low-molecular-weight PMEA has a weight-average molecular weight (Mw) of 1,700 to 280,000 as measured by gel permeation chromatography. In another embodiment of the present invention, the low-molecular-weight PMEA has a weight-average molecular weight (Mw) of 1,700 to 80,000 as measured by gel permeation chromatography. In another embodiment of the present invention, when the number-average molecular weight (Mn) of PMEA in the composition according to the present invention, measured by gel permeation chromatography (GPC), is greater than the polydispersity index of MEA measured by GPC in the present invention and is 3.0 or less, the PMEA is referred to as low-molecular-weight PMEA. In another embodiment of the present invention, the low-molecular-weight PMEA has a polydispersity index of 1.1 to 3.0 as measured by gel permeation chromatography. In another embodiment of the present invention, the low-molecular-weight PMEA has a polydispersity index of 1.2 to 2.5 as measured by gel permeation chromatography. In another embodiment of the present invention, the low-molecular-weight PMEA has a polydispersity index of 1.3 to 2.0 as measured by gel permeation chromatography. In another embodiment of the present invention, when the number-average molecular weight (Mn) of PMEA in the composition according to the present invention, measured by gel permeation chromatography, is greater than 100,000, the PMEA is referred to as high-molecular-weight PMEA. In another embodiment of the present invention, the high-molecular-weight PMEA has a number-average molecular weight (Mn) greater than 90,000 as measured by gel permeation chromatography. In another embodiment of the present invention, the high-molecular-weight PMEA has a number-average molecular weight (Mn) greater than 80,000 as measured by gel permeation chromatography for the composition according to the present invention. In another embodiment of the present invention, when the weight-average molecular weight (Mw) of PMEA in the composition according to the present invention, measured by gel permeation chromatography, is greater than 400,000, the PMEA is referred to as high-molecular-weight PMEA. In another embodiment of the present invention, the high-molecular-weight PMEA has a weight-average molecular weight (Mw) greater than 350,000 as measured by gel permeation chromatography. In another embodiment of the present invention, the high-molecular-weight PMEA has a weight-average molecular weight (Mw) greater than 300,000 as measured by gel permeation chromatography. In another embodiment of the present invention, the high-molecular-weight PMEA has a weight-average molecular weight (Mw) greater than 280,000 as measured by gel permeation chromatography.

To achieve the above objects, the present invention also provides a method for preparing an antibacterial or antifungal composition comprising a step of mixing methyl methacrylate liquid with methyl methacrylate powder.

In one embodiment of the present invention, the methyl methacrylate liquid is used in a state premixed with methoxyethyl acrylate.

To achieve the above objects, the present invention also provides a method for preparing an antibacterial or antifungal composition comprising steps of: (a) preparing, based on the total weight of the antibacterial or antifungal composition, 30 to 39.8 wt % of methyl methacrylate liquid; (b) mixing methyl methacrylate powder with the material obtained in step (a); and (c) forming a mixed resin by subjecting the material, obtained in step (b), to low-temperature polymerization.

In one embodiment of the present invention, step (a) further comprises a step of mixing the methyl methacrylate liquid uniformly with methoxyethyl acrylate to obtain a mixed solution.

To achieve the above objects, the present invention also provides a medical device including the above-described composition.

In one embodiment of the present invention, the composition is included to coat the surface of the medical device. In another embodiment of the present invention, the medical device is an internal restorative material, a temporary dental restorative material, a permanent dental restorative material, a pediatric dental restorative material, dentures, a dental implant, a mouthpiece, an occlusal stabilization splint, a night guard, an intraoral appliance, an activator, a snoring device, or an in vitro appliance.

To achieve the above objects, the present invention also provides a method for manufacturing a medical device comprising steps of: (a) preparing the above-described composition; (b) forming a mixed resin by subjecting the composition of step (a) to low-temperature polymerization; and (c) applying the resin obtained in step (b) to a medical device.

In one embodiment of the present invention, the medical device is an internal restorative material, a temporary dental restorative material, a permanent dental restorative material, a pediatric dental restorative material, dentures, a dental implant, a mouthpiece, an occlusal stabilization splint, a night guard, an intraoral appliance, an activator, a snoring device, or an in vitro appliance.

To achieve the above objects, the present invention also provides a method for manufacturing a medical device comprising steps of: (a) preparing, based on the total weight of the antibacterial or antifungal composition, 30 to 39.8 wt % of methyl methacrylate liquid; (b) mixing methyl methacrylate powder with the material obtained in step (a); (c) forming a mixed resin by subjecting the material, obtained in step (b), to low-temperature polymerization; and (d) polishing the mixed resin with SiC sandpaper.

In one embodiment of the present invention, step (a) further comprises a step of mixing the methyl methacrylate liquid uniformly with methoxyethyl acrylate to obtain a mixed solution. In another embodiment of the present invention, the methoxyethyl acrylate in step (a) has a number-average molecular weight (Mn) of 1,000 to 100,000. In another embodiment of the present invention, the methoxyethyl acrylate in step (a) has a weight-average molecular weight (Mw) of 1,500 to 400,000. In another embodiment of the present invention, the methoxyethyl acrylate in step (a) is used in an amount of 1 to 20 wt % based on the total weight of the composition. In another embodiment of the present invention, the methyl methacrylate powder in step (b) is used in an amount of 42 to 59.8 wt % based on the total weight of the antibacterial or antifungal composition. In another embodiment of the present invention, step (a) further comprises a step of continuously stirring for 12 hours to 48 hours. In another embodiment of the present invention, step (a) further comprises a step of continuously stirring for 18 hours to 36 hours. In another embodiment of the present invention, step (a) further comprises a step of continuously stirring for 20 hours to 28 hours. In another embodiment of the present invention, step (b) further comprises a step of stirring for 5 seconds to 30 seconds. In another embodiment of the present invention, step (b) further comprises a step of stirring for 8 seconds to 22 seconds. In another embodiment of the present invention, step (b) further comprises a step of stirring for 10 seconds to 18 seconds.

The effects of the present invention are as follows. The present invention may provide a composition which has improved mechanical properties, such as improved flexural strength, elastic modulus and hardness, as well as improved antibacterial or antifungal activity, by containing methoxyethyl acrylate at a predetermined ratio.

When the composition of the present invention is applied to a medical device, it may exhibit antibacterial or antifungal activity together with an antifouling action, and the medical device may prevent inflammatory reactions caused by infection with bacteria or fungi and exhibit excellent antibacterial activity.

### BRIEF DESCRIPTION OF THE DRAWINGS

FIGS. 1A and 1B depict graphs showing the mechanical properties of the composition according to the present invention. FIG. 1A: flexural strength; FIG. 1B: elastic modulus; and FIG. 1C: Vickers hardness.

FIG. 2 shows SEM images (100×, 200×, 500×, and 1.00K× magnifications) of the fracture surface of a specimen comprising the composition according to the present invention. White arrows indicate pores.

FIGS. 3A and 3B depict graphs showing the wettability and protein adsorption of a specimen comprising the composition according to the present invention. FIG. 3A is a graph showing the contact angle, and FIG. 3B is a graph showing the amount of adsorbed BSA.

FIGS. 4A, 4B, 4C, 4D, 4E and 4F show fungal and bacterial adhesion and viability on the composition according to the present invention. FIGS. 4A and 4D are representative live/dead staining images of Streptococcus mutans (FIG. 4A) and Candida albicans (FIG. 4D) adhered to the surface of the specimen; and the scale bar is 500 μm. FIGS. 4B and 4E show the results of WST assay for Streptococcus mutans (FIG. 4B) and Candida albicans (FIG. 4E) adhered to the surface (P<0.001). FIGS. 4C and 4F are scanning electron images showing that each of Streptococcus mutans and Candida albicans adheres to the surface (5000× and 2000× magnifications).

FIGS. 5A, 5B and 5C show the results of analysis of saliva-derived biofilm. FIG. 5A shows representative live/dead staining image of the biofilm adhered to the surface of a specimen comprising the composition according to the present invention, and FIGS. 5B and 5C are graphs showing the results of quantitative analysis of the biofilm thickness and the biofilm biomass, respectively (P<0.001).

FIGS. 6A, 6B and 6C depict graphs showing the mechanical durability of the composition according to the present invention. FIG. 6A: flexural strength; FIG. 6B: elastic modulus; and FIG. 6C: Vickers hardness.

FIGS. 7A, 7B and 7C depict images and graphs showing the biochemical durability of the composition according to the present invention. FIG. 7A shows representative live/dead staining images of the biofilm adhered to the surface after static immersion aging, FIG. 7B shows the results of quantitative analysis of the biofilm thickness, and FIG. 7C shows the results of quantitative analysis of the biofilm biomass (P<0.05).

FIG. 8 shows confocal laser microscope images of Preparation Example 1 and Comparative Example 2 tagged with rhodamine. Here, the scale bar is 500 μm.

FIGS. 9A and 9B depict graphs showing surface gross (FIG. 9A) and direct transmittance (FIG. 9B) (P<0.001).

FIGS. 10A, 10B and 10C depict graphs showing the amounts of adsorbed protein, that is, adsorbed BSA, on Comparative Example 1 (FIG. 10A), Preparation Example 1 (FIG. 10B) and Comparative Example 2 (FIG. 10C) (** P<0.01, *** P<0.001).

### DETAILED DESCRIPTION

Hereinafter, the present invention will be described in more detail with reference to examples. These examples are only for illustrating the present invention in more detail, and it will be apparent to those of ordinary skill in the art that the scope of the present invention according to the subject matter of the present invention is not limited by these examples.

### Synthesis of Poly(2-Methoxyethyl Acrylate) Polymer

### Preparation of Materials for Synthesizing Poly(2-Methoxyethyl Acrylate) Polymer

Methacryloyl thiocarbamoyl rhodamine-B (RhB) was purchased from Polysciences, Inc. Methoxyethyl acrylate (MEA), methyl mercaptopropionate (MMP) and reagent grade solvents were purchased from Fisher Scientific. 2,2′-azobisisobutyronitrile (AIBN) was purchased from Sigma-Aldrich and recrystallized from hot methanol before use. Gel permeation chromatography (GPC) was performed on a Shimadzu instrument at a rate of 1 ml/min using THF as a solvent. As the calibration standard in the instrument, poly(methyl methacrylate) from 1,000,000 to 92 was used. Gel permeation chromatography (GPC) analysis was performed using a Waters 1515 HPLC instrument equipped sequentially with Waters Styragel (7.8×300 mm) HR 0.5, HR 1 and HR 4 columns, and detection was performed using a differential refractometer (RI). ¹H NMR was performed using Varian MR400 (400 MHz) and Bruker 600 NMR, and data were analyzed using MestReNova software.

### Synthesis of Poly(2-Methoxyethyl Acrylate) (PMEA) Using Chain Transfer Agent

In a round bottom flask, methoxyethyl acrylate, a chain transfer agent, that is, methyl mercaptopropionate (MMP), and AIBN were dissolved in acetonitrile to give a monomer concentration of about 2 M. The reaction mixture was sealed, purged with nitrogen gas for 45 min, and then immersed in an oil bath at 70° C. After the reaction solution was stirred at 70° C. for 16 hours, the polymerization was stopped by cooling the reaction solution in a dry ice/acetone bath, and the reaction solution was exposed to air. The solvent was evaporated and the remaining solution was added dropwise to cold hexane with rapid stirring. The hexane layer was decanted, and the viscous polymer was re-dissolved in a small amount of dichloromethane and added dropwise to cold hexane with rapid stirring. The hexane layer was decanted, and the polymer was dried under vacuum for 24 hours to obtain a viscous polymer. The conversion rate and polymerization degree were analyzed by NMR based on the relative proportion of protons of the chain transfer agent and the relative proportion of the polymer. The ratio between the monomer and the chain transfer agent was varied to obtain PMEA polymers with various molecular weights.

TABLE 1

| | Moles of MEA | | | |
|---|---|---|---|---|
| | monomer | Moles MMP | Moles of AIBN | Yield |
| PMEA-1a | 0.11 | 0.011 | 0.002 | 97% |
| PMEA-2a | 0.11 | 0.0011 | 0.0002 | 83% |
| PMEA-3a | 0.11 | 0.00011 | 0.00002 | 73% |
| PMEA-4a | 0.11 | — | | 69% |
| PMEA-1 | 0.35 | 0.035 | 0.007 | 95% |
| PMEA-2 | 0.35 | 0.0035 | 0.0007 | 93% |
| PMEA-3 | 0.35 | 0.00035 | 0.00007 | 88% |
| PMEA-4 | 0.35 | 0 | 0.0035 | 85% |

Among them, PMEA-1, PMEA-2, PMEA-3 and PMEA-4 were used in Preparation Example 1 and the Experimental Example.

### Polymer Synthesis for Short-Chain Polymers Using Rhodamine-B Tag

The protocol for PMEA-Rh polymer synthesis was performed in exactly the same manner as that for short-chain polymer synthesis by adding methacryloyl thiocarbamoyl rhodamine-B (0.0000385 mol, 0.01 mol %) to a trace amount of a fluorescent tag.

TABLE 2

| Polymer | Mₙ NMR | Mₙ GPC | M_{w} GPC | Ðₘ |
|---|---|---|---|---|
| PMEA-1a | 1700 | 1680 | 2200 | 1.3 |
| PMEA-2a | 8100 | 9900 | 19000 | 1.9 |
| PMEA-3a | 38000 | 59200 | 164000 | 2.8 |
| PMEA-4a | — | 83000 | 400000 | 5.0 |
| PMEA-1 | 1400 | 1200 | 2000 | 1.6 |
| PMEA-2 | 10300 | 13700 | 18000 | 1.3 |
| PMEA-3 | 67800 | 76700 | 148000 | 1.9 |
| PMEA-4 | — | 116000 | 412000 | 3.5 |
| PMEA-1RhB | 1270 | 1640 | 2300 | 1.4 |
| PMEA-4RhB | — | 30000 | 210000 | 7.0 |

### EXAMPLES

Materials

In the present invention, self-curing acrylic resin for orthodontic appliances (Ortho-Jet, Lang Dental Manufacturing Co. Inc.) was used. Specimen were prepared by mixing poly(methyl methacrylate) (PMMA) and poly(2-methoxyethyl acrylate) (PMEA) together. As shown in Tables 1 and 2 above, PMEA-1, PMEA-2, PMEA-3 and PMEA-4 having different molecular weights were used for different specimens. More specifically, specimens, each comprising a mixture of PMEA and PMMA, were prepared according to the equation “PMEA/(PMEA+MMA powder+MMA liquid)” so that the proportions of PMEA in the specimens were 0 wt % (control), 3 wt %, 5 wt % and 10 wt %, respectively, as shown in Table 3 below.

TABLE 3

| | PMEA (MEA)-based | | |
|---|---|---|---|
| | acrylic resin, wt % | | PMEA (MEA), |
| Groups | MMA powder | MMA liquid | wt % |
| Control | 60.0 | 40.0 | 0 |
| 3% PMEA (MEA) | 58.2 | 38.8 | 3.0 |
| 5% PMEA (MEA) | 57.0 | 38.0 | 5.0 |
| 10% PMEA (MEA) | 54.0 | 36.0 | 10.0 |

Specimen Preparation and Evaluation of Mechanical Properties

Methyl methacrylate (MMA) powder was mixed with methyl methacrylate (MMA) liquid) at a mass ratio of 3:2. First, the PMEA (MEA) polymer was uniformly mixed with methyl methacrylate liquid with continuous stirring for 24 hours. Using standardized polyacetal resin molds, specimens for each experiment were prepared to have various shapes (disk or bar shape) and sizes. The mixed solution of PMEA (MEA) and methyl methacrylate liquid was added to methyl methacrylate powder, and the mixture was stirred for 15 seconds, and then subjected to low-temperature polymerization (60° C., 4.0 bar, 15 min, air press unit, Sejong Dental), and then poured into a mold (disk or bar shape). The specimens were polished with SiC sandpaper (up to 2000 grit). Before testing, all the polymerized specimens were stored in distilled water at 37° C. for 48 hours according to ISO standards. Specimens for 10% PMEA-3 and 10% PMEA-4 were not prepared due to their fast curing.

Mechanical properties were evaluated according to ISO 20795-2. Specimens were prepared in dimensions of 3.3 mm (height)×10 mm (width)×25 mm (length). A universal tester (Model 3366, Instron) was used for the three-point bending test, and the flexural strength and elastic modulus of each specimen were measured at a span length of 50 mm and a crosshead speed of 5 mm/min. The flexural strength and the elastic modulus were calculated according to the standard equations defined in ISO. The Vickers hardness of each specimen was measured for 30 seconds using a durometer (DMH-2, Matsuzawa Seiki Co. Ltd.) at a test load of 300 gf (2.94 N). The average value for each specimen was calculated from the results of measurements at three points.

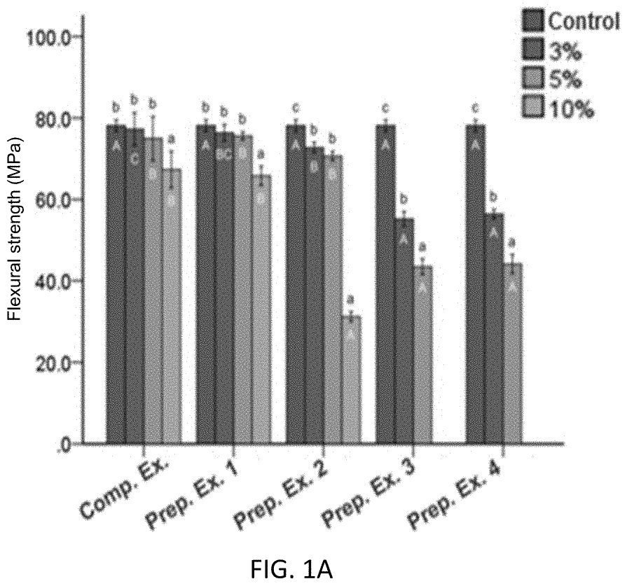

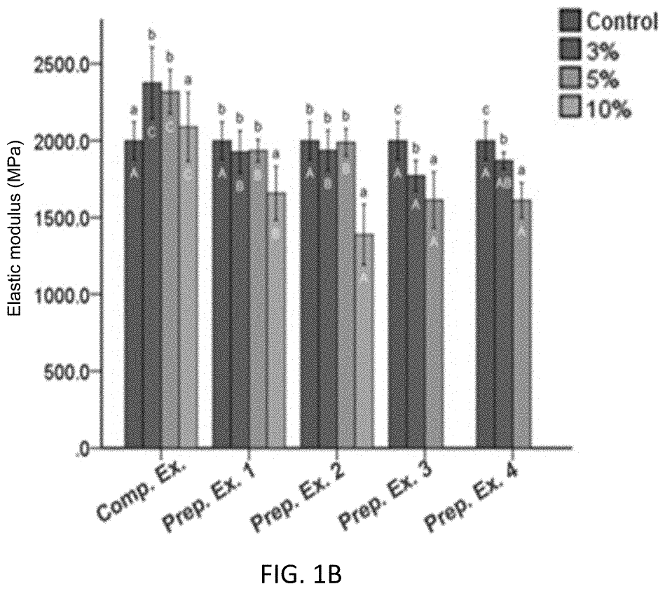

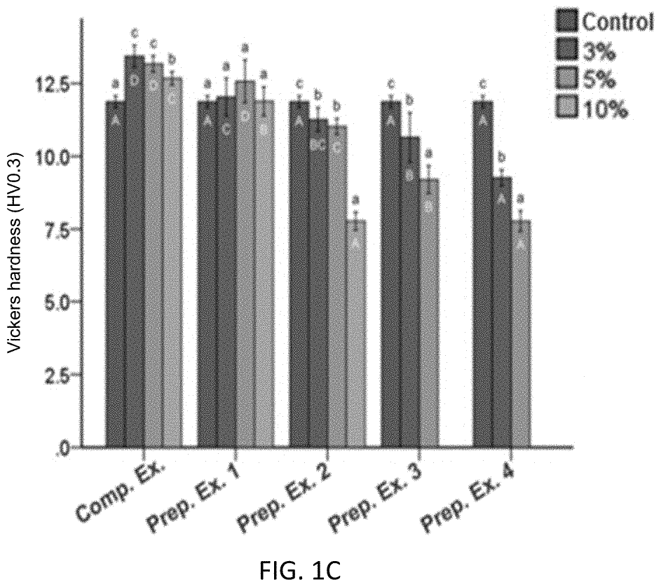

As a result, as shown in FIGS. 1A, 1B and 1C, it was possible to confirm the mechanical properties of the resins containing PMMA. It was observed that flexural strength (FIG. 1A), elastic modulus (FIG. 1B) and Vickers hardness (FIG. 1C) tended to decrease as the amount of PMEA increased. However, the 3% PMEA specimen and the 5% PMEA specimen showed significantly higher elastic modulus and Vickers hardness values than the control specimens, and the flexural strengths thereof did not significantly decrease, indicating that these specimens showed ideal mechanical properties. The mechanical properties of the 3% and 5% PMEA-1 specimens did not decrease, but the mechanical properties of the 10% PMEA-1 specimen significantly decreased. The flexural strengths of the 3% PMEA-3 and 3% PMEA-4 specimens were significantly lower, and the 5% PMEA-3 and 5% PMEA-4 specimens showed the value corresponding to the ISO standard. In addition, it was shown that, as the molecular weight of PMEA increased, the mechanical properties significantly decreased in order from PMEA-1 to PMEA-4 irrespective of the content of PMEA. Subsequent experiments were performed using the control, MEA, PMEA-1 and PMEA-4 specimens selected depending on the mechanical properties and protein adsorption test results (P<0.05).

In the following experiment, a composition containing low-molecular-weight PMEA and PMMA was set as Preparation Example 1. A composition containing only PMMA was set as a control, and a composition containing only MEA was set as Comparative Example 1. In the present invention, MEA (molecular weight=130.14) commercially available from Sigma was used. In the present invention, a composition containing high-molecular-weight PMEA and PMMA was set as Comparative Example 2 (see Table 4).

TABLE 4

| | Composition |
|---|---|
| Preparation | Composition containing low-molecular- |
| Examples 1 to 3 | weight PMEA and PMMA |
| Control | Composition containing only PMMA |
| Comparative | Composition containing only MEA |
| Example 1 | |
| Comparative | Composition containing high-molecular- |
| Example 2 | weight PMEA and PMMA |

### Experimental Example

Morphological Characteristics

In order to characterize the specimen containing the composition according to the present invention, bar-shaped specimens, each having a size of 3.3 mm (height)×10 mm (width)×25 mm (length), were fractured using a computer-controlled universal testing machine. The fractured surface of each specimen was coated with 5-nm Pt using an ion coater (ACE600; Leica) and then examined and imaged using a field emission scanning electron microscope (FE-SEM; Merin, Carl Zeiss, Oberkochen, Germany) at 5 kV.

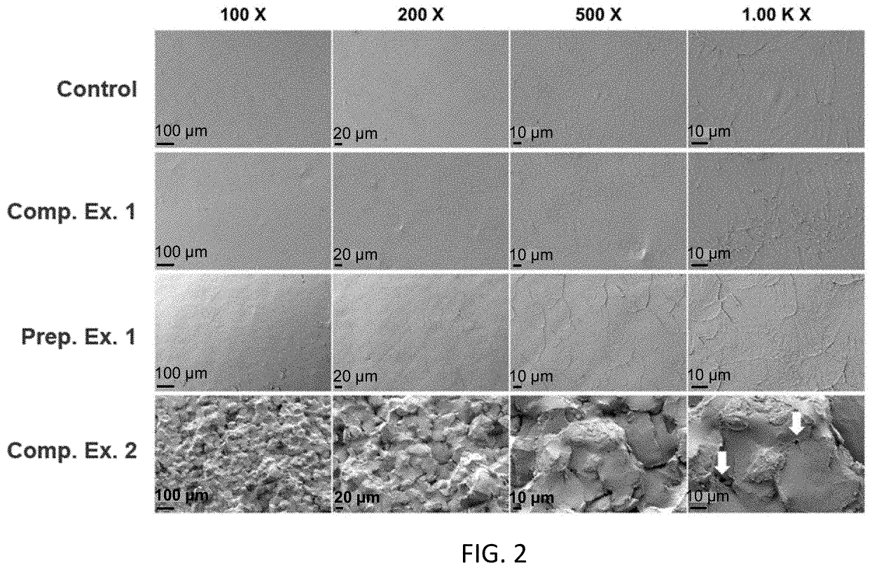

As a result, there was no noticeable difference between the specimen of Comparative Example 1 and the control specimen, and these specimens showed a smooth fracture surface (FIG. 2). The specimen of Preparation Example 1 showed a slightly protruding texture, but the entire fracture surface thereof was maintained flat. Unlike the other specimens, the surface of the specimen of Comparative Example 2 showed a high level of unevenness, and pores having various sizes were observed at 1.00K× (white arrows).

Wettability

Disk-shaped specimens (diameter: 15 mm, and thickness: 2 mm) were prepared using a standardized polyacetal resin mold. After drying of each specimen, 5 μL of distilled water was dropped onto the surface of each specimen, and after 10 seconds, the contact angle between the water and the surface was measured using a contact angle goniometer (SmartDrop, Femtobiomed Inc.). The measurement was repeated twice for each specimen and the average value was recorded.

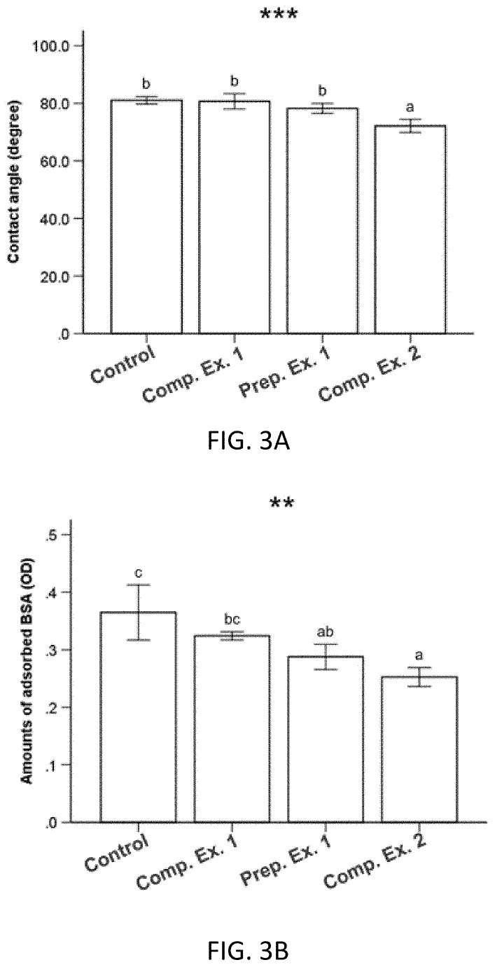

The results showed that the contact angle slightly decreased (meaning an increase in wettability) as the molecular weight increased (FIG. 3A). There was still no significant difference between the control specimen and the specimens of Comparative Example 1 and Preparation Example 1. Comparative Example 2 showed the lowest contact angle (72.13±2.29), suggesting that it showed the highest wettability (P<0.001).

Protein Adsorption

Disk-shaped specimens (diameter: 15 mm, and thickness: 2 mm) were prepared and immersed in fresh phosphate buffered saline (PBS; Gibco) at room temperature for 1 hour. Then, each specimen was immersed in bovine serum albumin (BSA; Pierce Biotechnology) broth (2 mg of protein/mL of PBS, 100 μL). After incubation for 4 hours in 5% CO₂ at 37° C., protein that did not adhere to the specimen was removed by washing twice with PBS. Next, the amount of protein adhered to each specimen was measured using micro-bicinchoninic acid (200 μL; Micro BCA™ Protein Assay Kit, Pierce Biotechnology), followed by incubation at 37° C. for 30 minutes. The amount of protein adsorbed to the surface was quantified based on the optical density (OD) at 562 nm, and was measured using a microplate reader (Epoch, BioTek Instruments).

The OD value for the BSA adsorption of the control specimen was higher than those of the other experimental groups (FIG. 3B). It was observed that protein adsorption decreased as the molecular weight increased. Comparative Example 2 showed the lowest protein adsorption (0.25±0.016), which did not significantly differ from that of Preparation Example 1 (P<0.01).

Fungal and Bacterial Adhesion and Viability

Disk-shaped specimens were prepared (diameter: 10 mm, and thickness: 2 mm). Fungal and bacterial analyses were performed using Candida albicans (Korean Collection for Oral Microbiology (KCOM) 1301) and Streptococcus mutans (ATCC 25175). A fungal or bacterial suspension (1 mL, 1×10⁸ cells/mL) was added to each specimen, and then incubated in 24-well plates at 37° C. for 24 hours. After incubation, non-adherent fungi or bacteria were removed by washing twice with PBS. Bacteria adhered to the surface of each specimen were harvested by sonication (SH-2100, Saehan Ultrasound) in brain heart infusion (BHI, 1 mL) for 5 minutes.

Microbial Viability Assay Kit-WST (Dojindo, Kumamoto, Japan) was used as a colorimetric indicator in direct proportion to the number of living cells according to the manufacturer's technical manual. A coloring reagent (10 μl) was added to the harvested bacterial suspension (190 μl), which was then incubated in a 96-well plate at 37° C. for 2 hours, and then the absorbance at 450 nm was measured using a microplate reader (Epoch, BioTek Instruments). The results are presented as the average of three experiments.

A live/dead cell viability kit (Molecular Probes, Eugene, Oreg., USA) was used to test the viability of adherent bacteria according to the manufacturer's protocol. Candida albicans and Streptococcus mutans were incubated in the same manner as described above. The stained specimens were observed with a confocal laser microscope (CLSM; LSM880, Carl Zeiss, Thornwood, N.Y., USA). Live bacteria appeared green, and dead bacteria appeared red.

For microscopic examination, bacteria adhered to each specimen were fixed with 2% glutaraldehyde-paraformaldehyde in 0.1M PBS at room temperature for at least 30 minutes. Each specimen was post-fixed with 1% OsO₄ in 0.1M PBS for 2 hours, dehydrated in gradually increasing ethanol concentrations, treated with isoamylacetate, and then subjected to critical-point drying (LEICA EM CPD300; Leica, Wien, Austria). Next, each disk specimen was coated with 5-nm Pt using an ion coater (ACE600; Leica), and examined and imaged using a field emission scanning electron microscope (FE-SEM; Merin, Carl Zeiss, Oberkochen, Germany) at 7 kV.

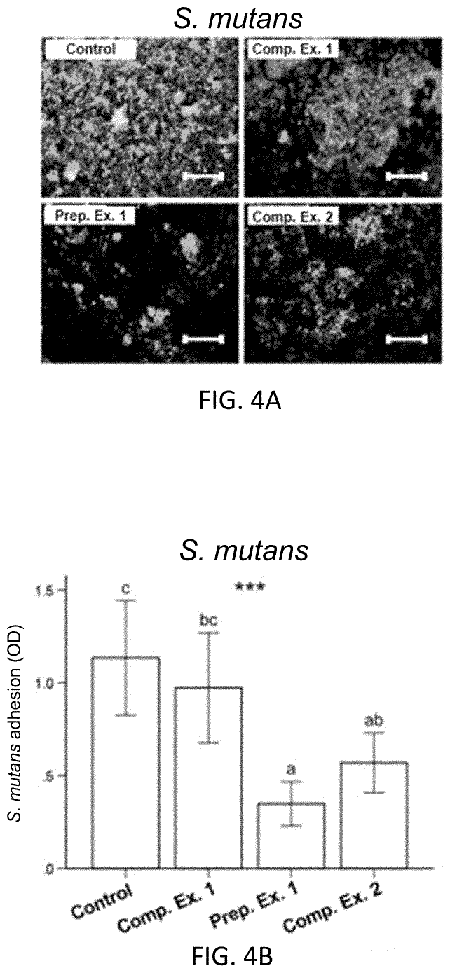

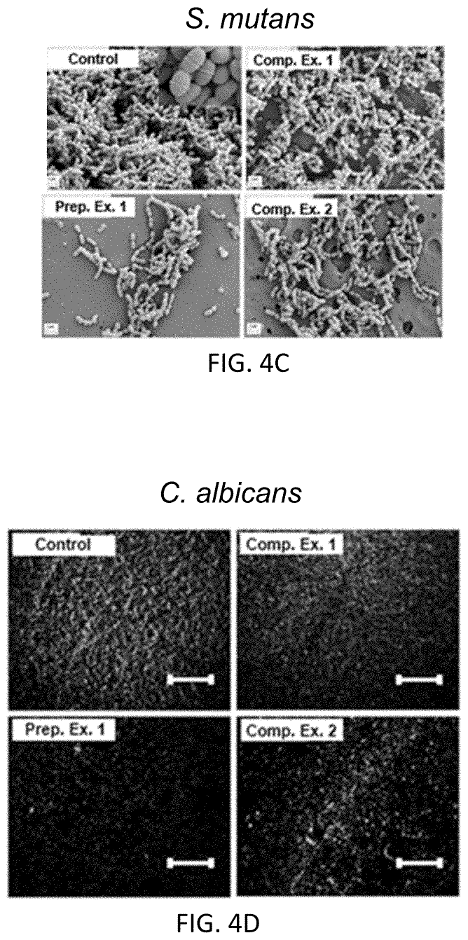

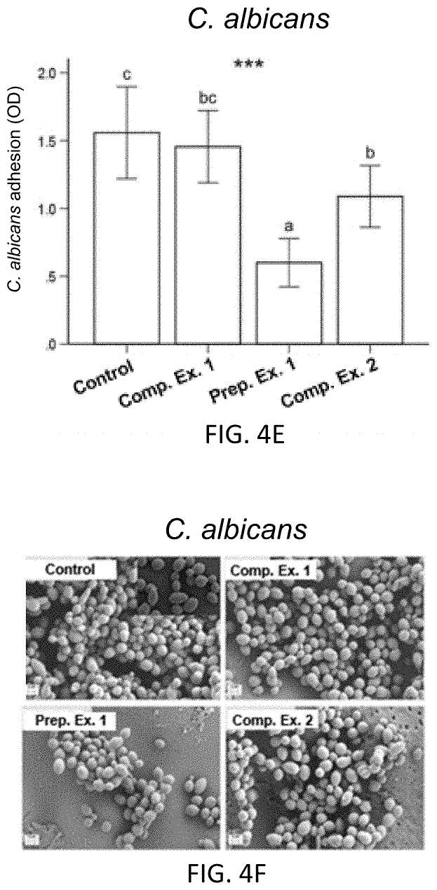

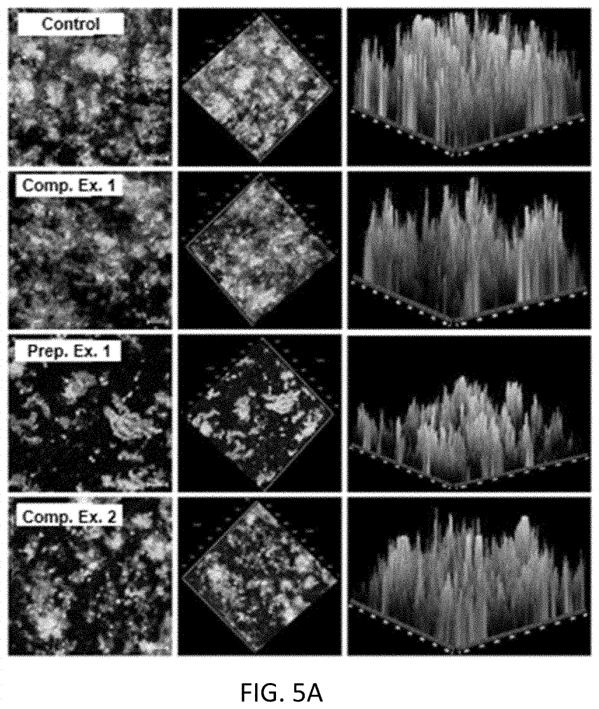

All the specimens were mainly covered with live bacteria (stained in green) (FIGS. 4A and 4D). The control specimen showed the strongest green fluorescence, and Preparation Example 1 showed less bacterial adhesion than the other groups. Water soluble tetrazolium salt (WST) assay (FIGS. 4B and 4E) indicated that the specimen of Preparation Example 1 showed the lowest OD value in both Candida albicans and Streptococcus mutans (P<0.001). Moreover, Comparative Example 1 showed less bacterial adhesion than the control, but the difference was not significant. These results were further confirmed by the FE-SEM images (FIGS. 4C and 4F).

Saliva-Derived Biofilm Model and Biomass Measurement

Human saliva was collected according to the procedure (2-2019-0049) approved by the institutional review committee of Yonsei University Dental Hospital (Seoul, Korea) in accordance with the Ethical Principles of the 64^{th} World Medical Association Declaration of Helsinki. Written consent was obtained from all participants prior to saliva donation. Human saliva samples obtained from six adults were mixed in equal proportions, and then diluted to 30% in sterile glycerol and stored at −80° C.

The biofilm model was incubated in McBain medium to simulate the salivary environment and obtain a stable microbial growth environment. The incubation medium (1.5 mL) was dropped onto each specimen (diameter: 10 mm, and thickness: 2 mm), and the biofilm was incubated at 37° C. for 48 hour. After 8 hours, 16 hours and 24 hours of incubation, additional incubation medium (1.5 mL) was added.

Each specimen was stained with a live/dead bacterial viability kit (Molecular Probes, Eugene, Oreg., USA) according to the manufacturer's protocol. Five sites were randomly selected under CLSM to observe the biofilm on the surface of each specimen. The biofilm thickness was measured using Zen software (Carl Zeiss) with respect to the vertical axis of the image. Average biomass was measured using the COMSTAT plugin (Denmark Technical University) with ImageJ software (NTH).

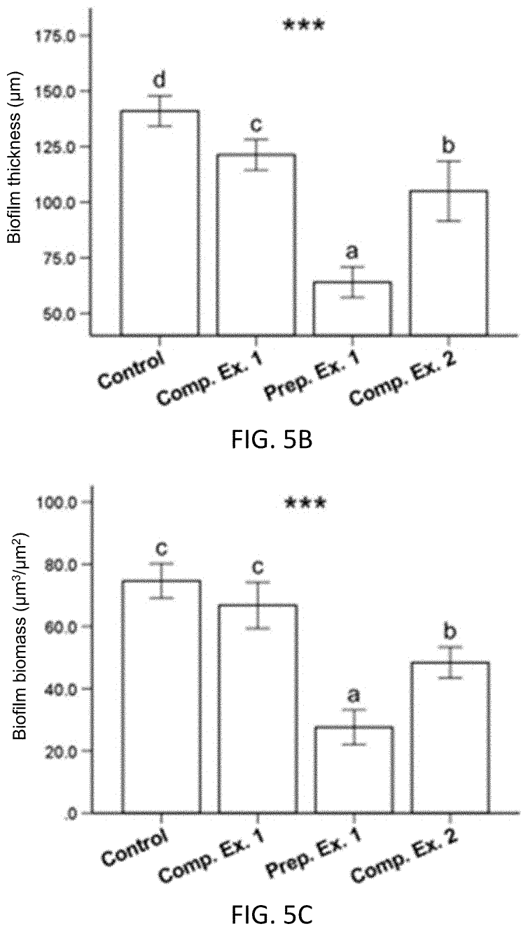

As shown in FIGS. 5A, 5B and 5C, it was possible to confirm the biofilm images, biofilm thicknesses and biomasses for several groups, which are consistent with those obtained for single bacteria (FIG. 5A). Biofilm biomass and thickness significantly decreased in the specimens of Preparation Example 1 and Comparative Example 2 specimens compared to the control specimen (FIGS. 5B and 5C) (P<0.001). The specimen of Comparative Example 2 showed less biofilm formation than the specimens of the control and Comparative Example 1. The specimen of Comparative Example 1 did not differ significantly from the control specimen in terms of biofilm biomass, but showed a significantly smaller biofilm thickness (P<0.001).

Durability Test

Durability analysis was performed using thermocycling aging for mechanical properties and using static immersion aging for long-term anti-biofilm effect. Each specimen was subjected to thermocycling equipment (Thermal Cyclic Tester, R & B Inc., Daejeon, Korea) at a dip time of 45 seconds and a transfer time of 5 seconds for 850 cycles, corresponding to 1 month. Thereafter, a mechanical test was performed in the same manner as described above. After immersing each disk-shaped specimen (diameter: 10 mm, and thickness: 2 mm) in distilled water at 37° C. for 7 days, the long-term anti-biofilm effect was analyzed. Saliva-derived biofilm model analysis was performed in the same procedure as previously mentioned.

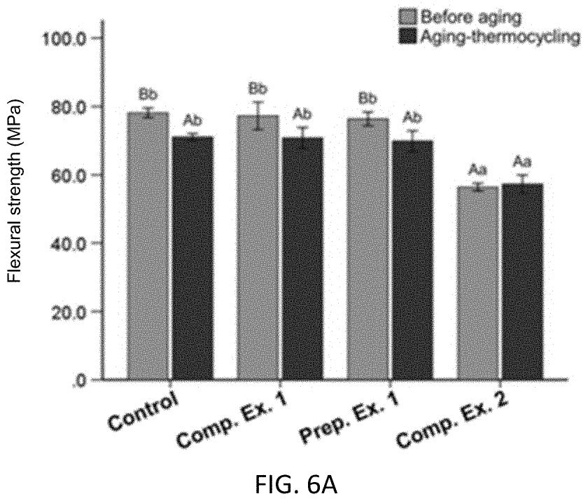

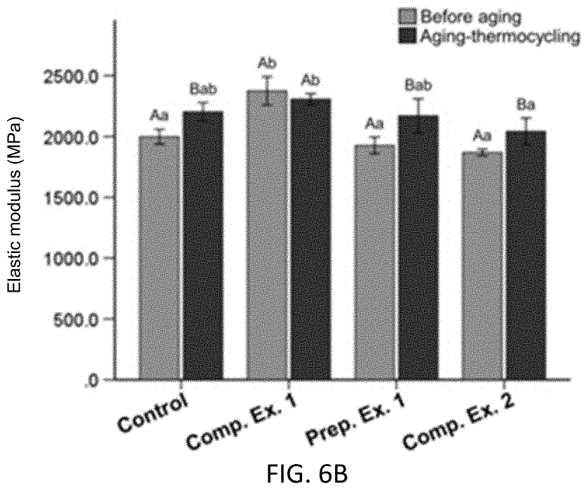

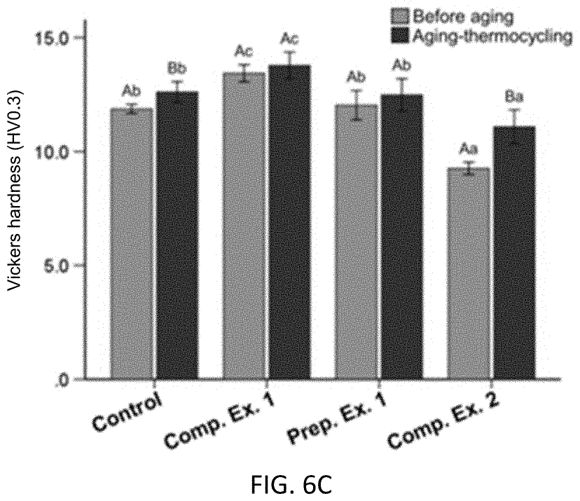

This test was performed under various aging conditions to evaluate mechanical and biochemical durability. The thermocycling-aged group showed mechanical properties similar to those of the group before aging (FIGS. 6A, 6B and 6C). In the case of the specimens of Comparative Example 1 and Preparation Example 1 after thermocycling aging, the elastic modulus significantly increased while the flexural strength greatly decreased. The flexural strength of the specimen of Comparative Example 2 did not change significantly even after aging, and the elastic modulus thereof significantly increased after aging. The specimens of the control and Comparative Example 2 showed a significantly increased Vickers hardness after aging, and the Vickers hardness of each of the specimen of Comparative Example 1 and the specimen of Preparation Example 1 between before and after aging did not significantly change (P<0.05).

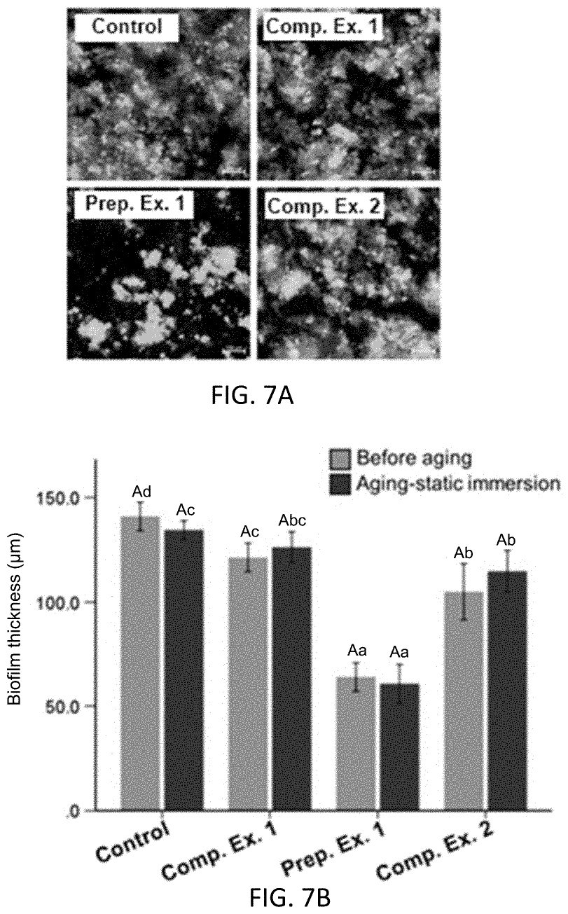

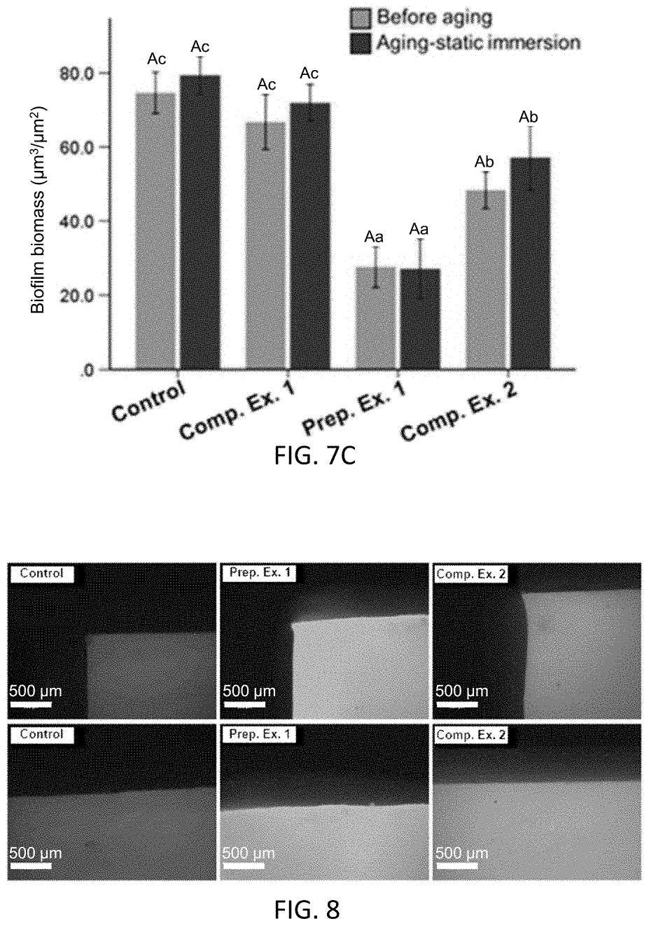

Static immersion aging was performed to evaluate biochemical durability. There was no significant difference between before and after aging in all the groups (FIGS. 7A, 7B and 7C). These groups showed a similar trend in biofilm formation after aging. Preparation Example 1 showed the smallest biofilm thickness and biomass (P<0.05).

Surface Separation

Rhodamine-tagged Preparation Example 1 and Comparative Example 2 were prepared. The rhodamine content was 0.1 mol %. Bar-shaped specimens were prepared in the same manner as described above. Each specimen was fractured using a computer-controlled universal testing machine, and the fractured surface was polished with SiC sandpaper (up to 2000 grit). Fluorescence images of the cross-sections were observed under CLSM.

FIG. 8 shows a separated layer of PMEA-PMMA resin. A clear and bright boundary was clearly observed in the specimen of Preparation Example 1 under a confocal laser microscope. The image was darker because a pure PMMA specimen was used as a control without rhodamine staining. Comparative Example 2 did not show a clearly separated surface.

Surface Gloss and Direct Transmittance

Disk-shaped specimens (diameter: 15 mm, and thickness: 2 mm) were prepared to measure the surface gloss and transparency. The surface gloss was measured using a calibrated infrared glossmeter (IG-330, Horiba) at an incident angle of 60°. The average value for each surface was calculated from 6 measurements.

An ultraviolet visible (UV/vis) spectrophotometer (Lambda 20, PerkinElmer) was used to analyze the direct transmittance (T %). Measurements were performed in the wavelength range of 400 to 780 nm with a data interval of 5 nm. The average T % value at 525 nm was used to represent the differences between materials.

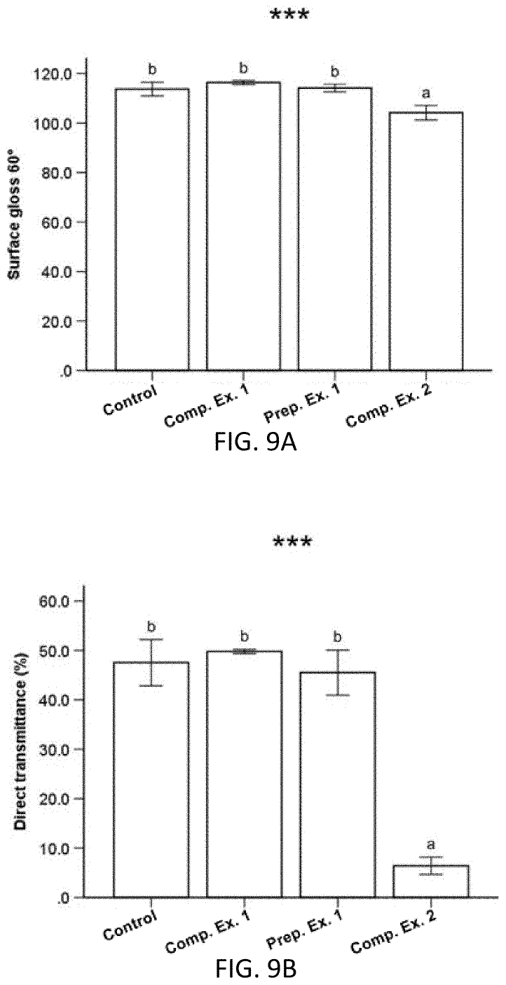

The specimens of Comparative Example 1 and Preparation Example 1 showed no significant difference from the control specimen in terms of both the surface gloss and the direct transmittance, whereas the specimen of Comparative Example 2 showed a significant decrease (FIGS. 9A and 9B) (P<0.001).

Contact Angles and Protein Adsorption of Comparative Example 1, Preparation Example 1 and Comparative Example 2

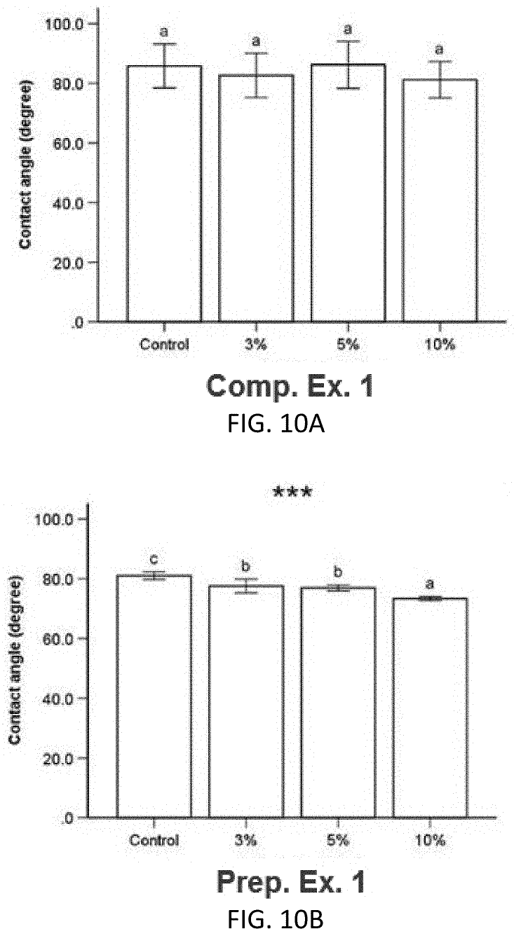

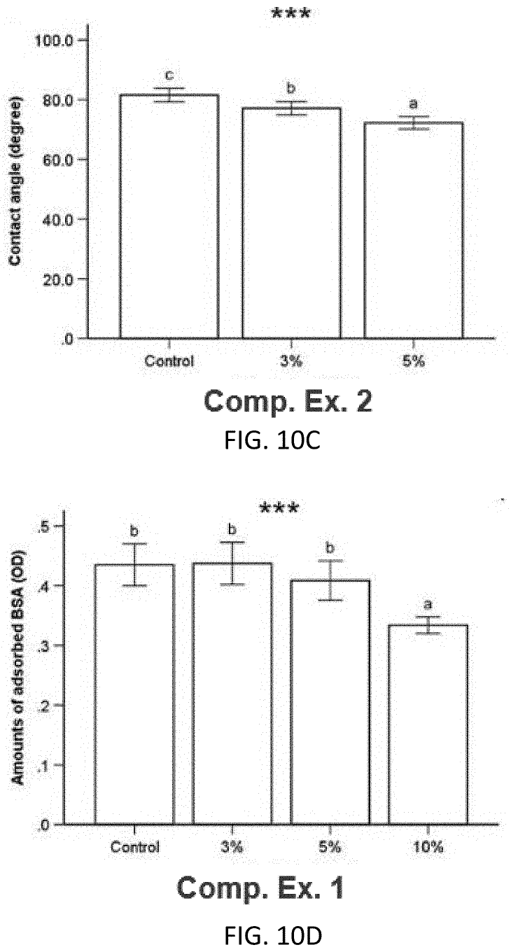

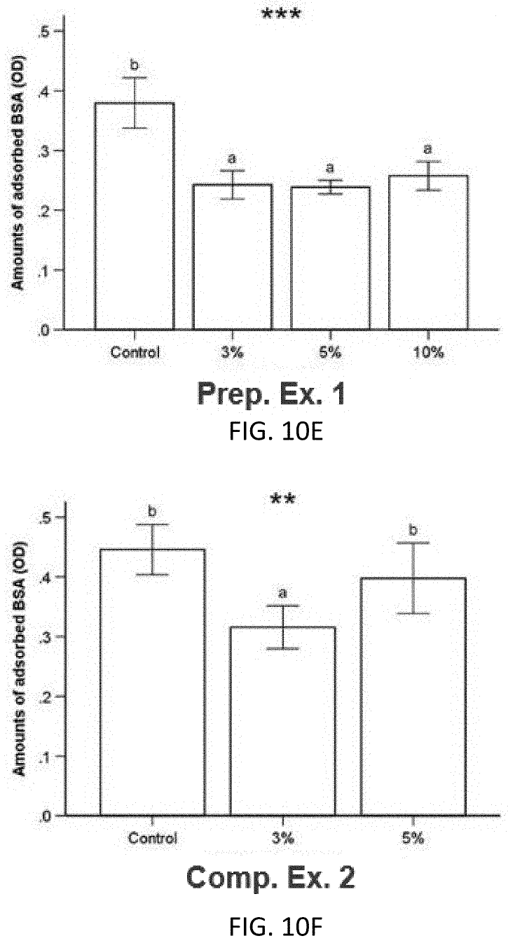

Referring to FIGS. 10A, 10B, 10C, 10D, 10E and 10F, it was observed that, as the content of MEA increased, Comparative Example 1 showed no change in the contact angle (see Table 3), and Preparation Example 1 and Comparative Example 2 showed a significant decrease in the contact angle (P<0.001). As shown in FIGS. 10A, 10B, 10C, 10D, 10E and 10F, the specimen of Comparative Example 1 containing 10% MEA (see Table 3) showed lower protein adsorption than the other specimens (P<0.001). All the 3%, 5% and 10% MEA specimens of Preparation Example 1 showed significantly decreased protein adsorption, and there was no significant difference between them (P<0.001). The 3% MEA specimen of Comparative Example 2 showed lower protein adsorption than the control and the 5% MEA specimen of Comparative Example 2 (P<0.01).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. An antibacterial or antifungal composition comprising methoxyethyl acrylate.

2. The antibacterial or antifungal composition of claim 1, further comprising poly(methyl methacrylate).

3. The antibacterial or antifungal composition of claim 2, wherein the poly(methyl methacrylate) is a mixture of methyl methacrylate powder and methyl methacrylate liquid.

4. The antibacterial or antifungal composition of claim 3, comprising, based on the total weight of the composition, 42 to 59.8 wt % of the methyl methacrylate powder, 30 to 39.8 wt % of the methyl methacrylate liquid, and 1 to 20 wt % of the methoxyethyl acrylate.

5. The antibacterial or antifungal composition of claim 4, comprising, based on the total weight of the composition, 45 to 59.2 wt % of the methyl methacrylate powder, 33 to 39.5 wt % of the methyl methacrylate liquid, and 1.5 to 18 wt % of the methoxyethyl acrylate.

6. The antibacterial or antifungal composition of claim 1, having antibacterial activity against at least one selected from the group consisting of Streptococcus mutans, Streptococcus sobrinus, Streptococcus sanguis, Streptococcus minor, Lactbacillus casei, Lactbacillus acidophilus, Porphyromonas gingivalis, Treponema denticola, Actinomyces naeslundii, Veillonella parvula, Actinomyces viscosus, and Actinomyces naeslundii.

7. The antibacterial or antifungal composition of claim 1, having antifungal activity against at least one selected from the group consisting of Candida albicans, Escherichia coli, Staphyloccoccus aureus, Pseudomonas aeruginosa, and Aspergillus niger.

8. The antibacterial or antifungal composition of claim 1, further comprising at least one selected from the group consisting of a stabilizer, a flame retardant, an antistatic agent, a softener, a reinforcing material, a filler, a fluorescent whitening agent, a lubricant, an inclusion reducer, a polycondensation catalyst, an antifoaming agent, an emulsifier, a thickener, and fragrances.

9. The antibacterial or antifungal composition of claim 1, further comprising an adhesive material.

10. The antibacterial or antifungal composition of claim 9, wherein the adhesive material is at least one selected from the group consisting of hydroxypropyl methylcellulose, hydroxyethyl cellulose, hydroxypropyl cellulose, polyvinyl alcohol, polyvinyl pyrrolidone, carbomer, and polyvinyl acetate resins.

11. A method for preparing an antibacterial or antifungal composition comprising a step of mixing methyl methacrylate liquid with methyl methacrylate powder.

12. The method of claim 11, wherein the methyl methacrylate liquid is used in a state premixed with methoxyethyl acrylate.

13. A medical device including the composition of claim 1.

14. The medical device of claim 13, wherein the composition is included to coat a surface of the medical device.

15. The medical device of claim 13, which is an internal restorative material, a temporary dental restorative material, a permanent dental restorative material, a pediatric dental restorative material, a denture, a dental implant, a mouthpiece, an occlusal stabilization splint, a night guard, an intraoral appliance, an activator, a snoring device, or an in vitro appliance.

16. A method for manufacturing a medical device comprising steps of:
(a) preparing the composition of claim 1;
(b) forming a mixed resin by subjecting the composition of step (a) to low-temperature polymerization; and
(c) applying the resin obtained in step (b) to a medical device.

17. The method of claim 16, wherein the medical device is an internal restorative material, a temporary dental restorative material, a permanent dental restorative material, a pediatric dental restorative material, a denture, a dental implant, a mouthpiece, an occlusal stabilization splint, a night guard, an intraoral appliance, an activator, a snoring device, or an in vitro appliance.
